(19) United States
(12) Patent Application Publication
(10) Pub. No.: US 2017/0135673 A1
(43) Pub. Date: May 18, 2017

(54) AN ULTRASOUND TRANSDUCER PROBE HAVING A CURVED IMAGING FACE

(71) Applicant: General Electric Company, Schenectady, NY (US)

(72) Inventors: Reinhold Bruestle, Zipf (AT); Stefan Denk, Zipf (AT); Scott William Easterbrook, Bainbridge Island, WA (US); Scott Daniel Cogan, East Lansing, MI (US); Charles Edward Baumgartner, Niskayuna, NY (US)

(21) Appl. No.: 14/941,688

(22) Filed: Nov. 16, 2015

(51) Int. Cl. A61B 8/00; G01S 7/52

(52) U.S. Cl. CPC A61B 8/4455; A61B 8/56; G01S 7/52079

(57) ABSTRACT

A transducer probe is presented. The transducer probe includes a housing having at least one curved surface at a first end. Further, the transducer probe includes a transducer unit including a plurality of electro-acoustic modules and configured to emit ultrasound signals towards a target volume. Also, the transducer probe includes at least one interconnect configured to electrically couple the transducer unit to a probe cable. Furthermore, the transducer probe includes an acoustic standoff positioned between the transducer unit and the curved surface of the housing and configured to propagate the ultrasound signals with minimal attenuation, minimal refraction, or both.

### BACKGROUND

Embodiments of the present specification relate generally to an ultrasound transducer probe, and more particularly to the ultrasound transducer probe having a curved imaging face.

Medical diagnostic ultrasound is an imaging modality that employs ultrasound waves to probe the acoustic properties of biological tissues and produces corresponding images. Particularly, ultrasound systems are used to provide an accurate visualization of muscles, tendons, and other internal organs to assess their size, structure, movement, and/or any pathological conditions using near real-time images. Moreover, owing to the ability to image the underlying tissues without the use of ionizing radiation, ultrasound systems find extensive use in angiography and prenatal scanning.

Conventional ultrasound systems employ a transducer probe that houses an array of transducer elements configured to emit ultrasound signals into a body or a target volume in a subject. Generally, conventional transducer probes include flat or curved transducer arrays for imaging different volumes of interest. By way of example, the flat transducer arrays are used in transducer probes employed for cardiac imaging. Further, the curved transducer arrays are used in transducer probes, for example, employed in abdominal imaging. More particularly, the curved transducer arrays are used to provide efficient ergonomics during imaging of curved surfaces such as breast tissues and/or an abdominal region in a patient.

Generally, the curved transducer arrays are formed by fabricating a large array in a flat configuration and subsequently bending the large array into its final form. Typically, the large array is in direct contact with beamforming electronics or an application specific integrated circuit (ASIC). Thus, while bending the large array, the beamforming electronics or ASIC are also bent along with the large array. Further, bending the beamforming electronics or ASIC may induce internal stresses, which in turn alter the functionality of the ASIC.

Alternatively, the curved transducer arrays may be fabricated by disposing multiple flat transducer segments onto a support structure having a curved shape. Particularly, the flat transducer segments may be positioned on a curved surface of the support structure to obtain curved transducer arrays or a curved imaging face. However, fabrication of such curved transducer arrays is a complicated and expensive process.

### BRIEF DESCRIPTION

In accordance with aspects of the present specification, a transducer probe is presented. The transducer probe includes a housing having at least one curved surface at a first end. Further, the transducer probe includes a transducer unit including a plurality of electro-acoustic modules and configured to emit ultrasound signals towards a target volume. Also, the transducer probe includes at least one interconnect configured to electrically couple the transducer unit to a probe cable. Furthermore, the transducer probe includes an acoustic standoff positioned between the transducer unit and the curved surface of the housing and configured to propagate the ultrasound signals with minimal attenuation, minimal refraction, or both.

In accordance with a further aspect of the present specification, a system for ultrasound imaging is presented. The system includes a transmit circuitry configured to generate ultrasonic pulses. Also, the system includes a transducer probe coupled to the transmit circuitry via a probe cable and including a housing having at least one curved surface at a first end. Further, the transducer probe includes a transducer unit including a plurality of electro-acoustic modules configured to receive the ultrasonic pulses and emit ultrasound signals towards a target volume. In addition, the transducer probe includes at least one interconnect configured to electrically couple the transducer unit to the probe cable. Moreover, the transducer probe includes an acoustic standoff positioned between the transducer unit and the at least one curved surface of the housing and configured to propagate the ultrasound signals with minimal attenuation, minimal refraction, or both.

In accordance with another aspect of the present specification, a method for forming an ultrasound probe is presented. The method includes forming a transducer unit having a flat top surface by arranging a plurality of electro-acoustic modules, wherein the transducer unit is configured to transmit ultrasound signals towards a target volume. Further, the method includes positioning the transducer unit having the flat top surface below at least one curved surface of a housing. Also, the method includes disposing an acoustic standoff between the transducer unit and the at least one curved surface of the housing to eliminate a distance of propagation for the ultrasound signals through air, wherein the acoustic standoff includes at least one curved top surface and a flat bottom surface.

### DRAWINGS

These and other features, aspects, and advantages of the present disclosure will become better understood when the following detailed description is read with reference to the accompanying drawings in which like characters represent like parts throughout the drawings, wherein:

FIG. 1 illustrates an ultrasound system for imaging a target volume in a subject, in accordance with aspects of the present specification;

FIG. 2 is a diagrammatical representation of a transducer probe having an acoustic standoff, in accordance with aspects of the present specification;

FIG. 3 is a perspective view of an electro-acoustic module for use in the transducer probe of FIG. 2, in accordance with aspects of the present specification;

FIG. 4 is a diagrammatical representation of a transducer array having a plurality of electro-acoustic modules of FIG. 3, in accordance with aspects of the present specification;

FIG. 5 is a diagrammatical representation of a portion of the transducer probe of FIG. 2 depicting the acoustic standoff, in accordance with aspects of the present specification; and

FIG. 6 is a flow chart illustrating a method for facilitating the propagation of ultrasound signals from a transducer unit to a housing of the transducer probe, in accordance with aspects of the present specification.

### DETAILED DESCRIPTION

As will be described in detail hereinafter, various embodiments of an ultrasound transducer probe and methods for fabricating the same are presented. In particular, the ultrasound transducer probe and methods presented herein employ an acoustic standoff over a flat transducer array to propagate ultrasound signals from the flat transducer array towards a subject being scanned. Moreover, the acoustic standoff is a water-like medium that aids in providing a curved shape to the transducer array, thereby enabling the propagation of sound waves from the flat transducer array onto a curved probe surface. In addition, the acoustic standoff includes a non-focusing and low attenuation material that aids in propagating the ultrasound signals with minimal energy loss and focusing effects. Additionally, use of the flat transducer array significantly reduces the cost and complexity of fabrication of the transducer probe, while a curved surface of the acoustic standoff provides ergonomic imaging capability for scanning desired volumes in the subject.

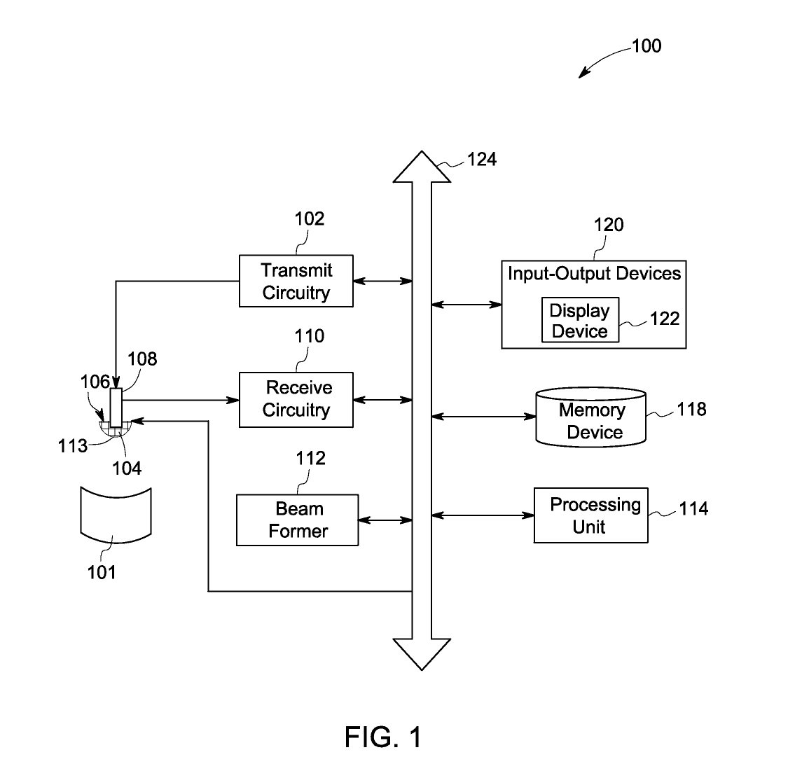

Although the following description includes embodiments relating to ultrasound imaging, these embodiments may also be implemented in other medical imaging systems that employ devices such as ultrasound and/or interventional probes during imaging. These systems, for example, may include magnetic resonance imaging (MRI) systems, computed-tomography (CT) systems, and systems that monitor targeted drug and gene delivery. Additionally, the systems may be used for accurate diagnosis and staging of coronary artery disease and monitoring of therapies including high-intensity focused ultrasound (HIFU), radiofrequency ablation (RFA), and brachytherapy. An exemplary environment that is suitable for practising various implementations of the present system is described in the following sections with reference to FIG. 1.

FIG. 1 illustrates an ultrasound system 100 for imaging a target volume 101 in a subject. In one embodiment, the ultrasound system 100 may be configured as a console system or a cart-based system. Alternatively, the ultrasound system 100 may be configured as a portable system, such as a hand-held, laptop-style and/or a smartphone-based system. For ease of description, the ultrasound system 100 is represented as a portable ultrasound system.

Further, in the present specification, the ultrasound system 100 is presented as being used to image a target volume 101 in biological tissues of interest. In one example, the target volume 101 may include cardiac tissues, liver tissues, breast tissues, prostate tissues, thyroid tissues, lymph nodes, vascular structures adipose tissue, muscular tissue, and/or blood cells. Alternatively, the system 100 may be employed for imaging non-biological materials such as manufactured parts, plastics, aerospace composites, and/or foreign objects within a body such as a catheter or a needle.

In certain embodiments, the system 100 includes transmit circuitry 102 that generates a pulsed waveform to drive a transducer array 104 of transducer elements 106 housed within a transducer probe 108. Particularly, the pulsed waveform drives the transducer array 104 of transducer elements 106 to emit ultrasonic pulses into the target volume 101. The transducer elements 106, for example, may include piezoelectric, piezoceramic, capacitive, and/or microfabricated crystals. At least a portion of the ultrasonic pulses generated by the transducer elements 106 is back-scattered from the target volume 101 to produce echoes that return to the transducer array 104 and are received by receive circuitry 110 for further processing. It may be noted that the terms “ultrasonic” and “ultrasound” may be used interchangeably in the following description.

Also, in the embodiment illustrated in FIG. 1, the receive circuitry 110 is coupled to a beamformer 112 that processes the received echoes and outputs corresponding radio frequency (RF) signals. Subsequently, a processing unit 114 receives and processes the RF signals in near real-time and/or offline mode. The processing unit 114 includes devices such as one or more general-purpose or application-specific processors, digital signal processors, microcomputers, microcontrollers, Application Specific Integrated Circuits (ASICs), Field Programmable Gate Arrays (FPGA), or other suitable devices in communication with other components of the system 100.

Moreover, in certain embodiments, the processing unit 114 also provides control and timing signals for configuring one or more imaging parameters for imaging the target volume 101 in the subject. Furthermore, in one embodiment, the processing unit 114 stores the delivery sequence, frequency, time delay, and/or beam intensity, for example, in a memory device 118 for use in imaging the target volume 101. The memory device 118 includes storage devices such as a random access memory, a read only memory, a disc drive, solid-state memory device, and/or a flash memory. In one embodiment, the processing unit 114 uses the stored information for configuring the transducer elements 106 to direct one or more groups of pulse sequences toward the target volume 101. Subsequently, the processing unit 114 tracks the displacements in the target volume 101 caused in response to the incident pulses to determine corresponding tissue characteristics. The displacements and tissues characteristics, thus determined, are stored in the memory device 118. The displacements and tissues characteristics may also be communicated to a medical practitioner, such as a radiologist, for further diagnosis.

In some embodiments, the processing unit 114 may be further coupled to one or more user input-output devices 120 for receiving commands and inputs from an operator, such as the medical practitioner. The input-output devices 120, for example, may include devices such as a keyboard, a touchscreen, a microphone, a mouse, a control panel, a display device 122, a foot switch, a hand switch, and/or a button. In one embodiment, the processing unit 114 processes the RF signal data to prepare image frames and to generate the requested medically relevant information based on user input. Particularly, the processing unit 114 may be configured to process the RF signal data to generate two-dimensional (2D) and/or three-dimensional (3D) datasets corresponding to different imaging modes.

Further, the processing unit 114 may be configured to reconstruct desired images from the 2D or 3D datasets. Subsequently, the processing unit 114 may be configured to display the desired images on the associated display device 122 that may be communicatively coupled to the processing unit 114. The display device 122, for example, may be a local device. Alternatively, in one embodiment, the display device 122 may be remotely located to allow a remotely located medical practitioner to access the reconstructed images and/or medically relevant information corresponding to the target volume 101 in the subject/patient. It may be noted that the various components of the ultrasound system 100 are communicatively coupled via a communication channel 124.

In a conventional transducer probe, the transducer array may be bent into a curved shape to improve ergonomics of application of the probe or improve field of view of the target volume. However, when the transducer array is bent, the beamforming electronics or ASICs are also bent along with the transducer array. Further, bending the beamforming electronics or the ASICs may induce internal stresses, which in turn alter the functionality of the beamforming electronics or the ASICs. Alternatively, the curved transducer arrays may be fabricated by disposing multiple flat transducer elements onto a support structure having a curved shape. However, fabrication of such curved transducer arrays is a complicated and expensive process.

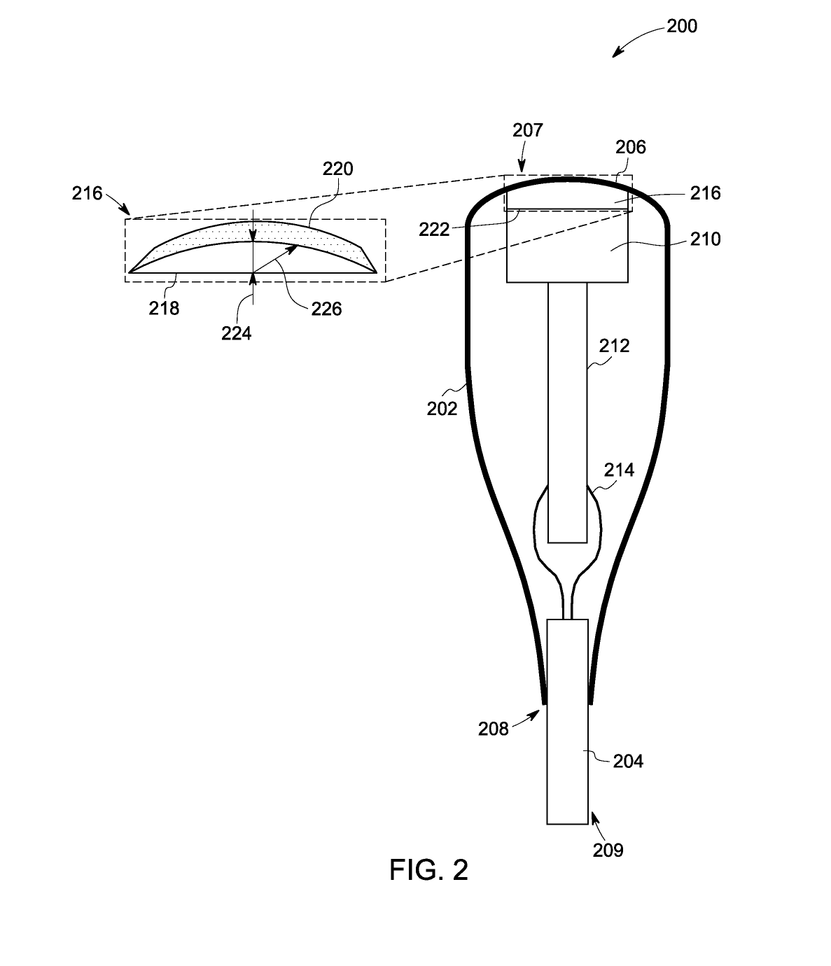

In accordance with aspects of the present specification, an exemplary transducer probe 108 configured to overcome the above shortcomings associated with currently available transducer probes is presented. In particular, the exemplary transducer probe 108 may employ an acoustic offset or acoustic standoff (see FIG. 2) to couple the transducer elements 106 and the target volume 101 in the subject. Particularly, the acoustic standoff may be disposed between the underlying flat transducer elements 106 and a housing that covers the transducer probe 108. More specifically, the acoustic standoff has a flat bottom surface and a curved top surface. Further, the flat bottom surface is coupled to the flat transducer elements 106, while the curved top surface is coupled to a curved surface 113 of the housing.

Moreover, the acoustic standoff may act as a medium for the ultrasound signals to propagate from the flat transducer elements 106 to the target volume 101. Also, the acoustic standoff may have one or more non-focusing and low attenuation materials that aid in propagating the ultrasound signals with minimal energy loss and minimal focusing effects. Certain exemplary configurations of a transducer probe having the acoustic standoff will be described in greater detail with reference to FIGS. 2-5.

Referring to FIG. 2, a diagrammatical representation of an exemplary transducer probe 200 having an acoustic standoff, in accordance with aspects of the present specification, is depicted. The transducer probe 200 may be representative of one embodiment of the ultrasound transducer probe 108 of FIG. 1. The ultrasound transducer probe 200 is described with reference to the components in FIG. 1. It may be noted that the terms “ultrasound transducer probe” and “transducer probe” may be used interchangeably.

The transducer probe 200 includes a housing 202 and a probe cable 204 coupled to the housing 202. The housing 202 may have one or more desired shapes depending upon a target volume 101 in a subject/body being scanned.

Further, the housing 202 may have a curved surface 206 at a first end 207 and an opening 208 at a second end 209 of the housing 202. The curved surface 206 may be a smooth closed surface that is used to contact the subject being scanned. In one example, the curved surface 206 may be formed using one or more materials that are used to provide mechanical protection at the first end 207 of the housing 202. In another example, the curved surface 206 may be formed using a smooth curving material that acts as a lens in the transducer probe 200. In addition, use of the curved surface 206 may allow optimal positioning of the transducer probe 200 on curved surfaces, such as the chest, breast, and/or abdominal regions of a patient. Moreover, the opening 208 at the second end 209 of the housing 202 is used to receive at least a portion of the probe cable 204 that is coupled to the housing 202, as depicted in FIG. 2.

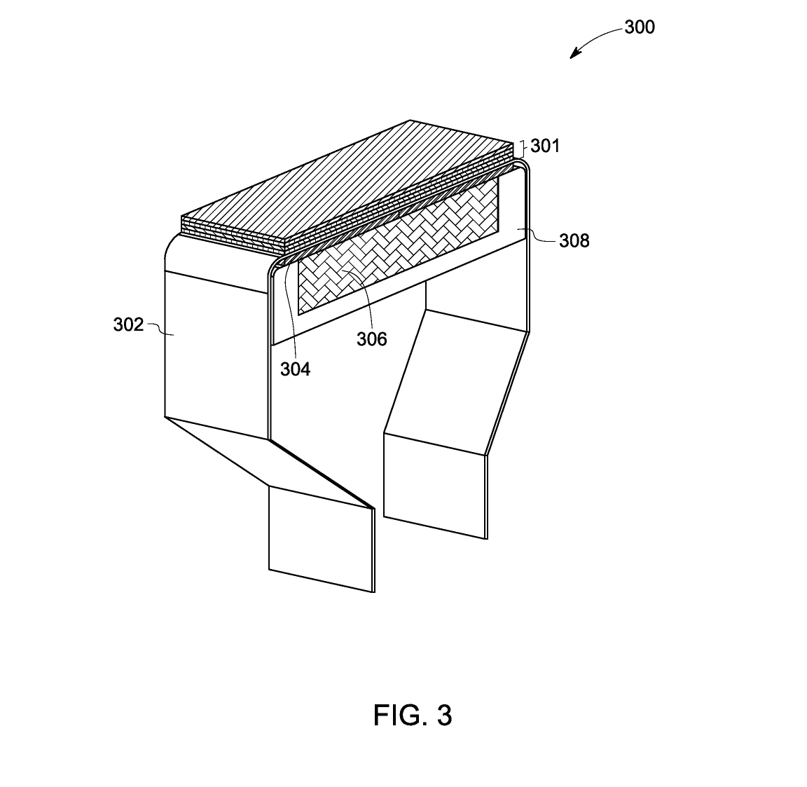

In a presently contemplated configuration, the housing 202 includes a transducer unit 210, one or more interconnects 212, and a cable 214. The transducer unit 210 may include a transducer array having one or more electro-acoustic modules that are coupled to a support structure (see FIG. 3). These electro-acoustic modules are used for emitting ultrasound signals towards the target volume 101 in the subject. It may be noted that the number of electro-acoustic modules included in the transducer array may vary depending upon the type of imaging and type of transducer design. For example, one electro-acoustic module may be included in a small transducer array for cardiac imaging. In another example, a plurality of electro-acoustic modules may be included in the transducer array for vascular imaging or obstetrics imaging. It may be noted that the terms “electro-acoustic modules” and “transducer elements” may be used interchangeably.

Further, the one or more interconnects 212 may be rigid or elongated flex interconnects that are flexible and adaptable to provide electrical connection between the transducer unit 210 and the cable 214 that protrudes from the probe cable 204. In one example, the one or more interconnects 212 may be used to communicate the ultrasonic/electrical pulses between a piezoelectric layer in the electro-acoustic modules and signal processing electronics within or outside the transducer probe 200.

In one exemplary embodiment, in addition to the transducer unit 210, the one or more interconnects 212, and the cable 214, the housing 202 may also include an acoustic standoff 216. In a presently contemplated configuration, the acoustic standoff 216 is positioned between the transducer unit 210 and the curved surface 206 of the housing 202. The acoustic standoff 216 may provide a propagation path for the ultrasound signals that propagate from the transducer unit 210 towards the subject being scanned. In one example, the acoustic standoff 216 may be employed to eliminate an air gap between the transducer unit 210 and the curved surface 206 of the housing 202. Also, the acoustic standoff 216 may provide mechanical protection to the electro-acoustic modules in the transducer unit 210.

Moreover, in one embodiment, the acoustic standoff 216 may have a flat bottom surface 218 and a curved top surface 220. Further, the flat bottom surface 218 of the acoustic standoff 216 is coupled to a top surface 222 of the transducer unit 210, while the curved top surface 220 of the acoustic standoff 216 is coupled to the curved surface 206 of the housing 202. In one example, ultraviolet (UV) light may be used to operatively couple the acoustic standoff 216 to the transducer unit 210 and the curved surface 206 of the housing 202. More particularly, in certain embodiments, UV light may be used to bond the acoustic standoff 216 to the transducer unit 210 and the curved surface 206 of the housing 202. In one embodiment, the acoustic standoff 216 may have a peak thickness (Tₚ) 224 in a range from about 0.5 mm to about 12 mm. Also, the acoustic standoff 216 may have a radius of curvature (ROC) 226 in a range from about 9 mm to about 60 mm.

Further, the acoustic standoff 216 includes one or more low attenuation materials that aid in propagating the ultrasound signals with minimal energy loss. It may be noted that the property of the acoustic standoff 216 that facilitates propagation of the ultrasound signals with minimal energy loss is referred to as “low attenuation” or “minimal attenuation” of the ultrasound signals. Typically, in the conventional probes, one or more acoustic lenses and other front face materials are used between the transducer unit and the curved surface of the probe for propagating the ultrasound signals from the transducer unit to the curved surface of the probe. However, these acoustic lenses and the front face materials may substantially attenuate the ultrasound signals and thereby cause significant energy loss in the ultrasound signals. Further, this energy loss in the ultrasound signals may negatively affect image quality. To circumvent these problems, in the exemplary probe 200, the acoustic standoff 216 having the low attenuation materials is used between the transducer unit 210 and the curved surface 206 to minimize the energy loss in the ultrasound signals, which in turn enhances the image quality.

Also, the acoustic standoff 216 includes one or more non-focusing materials that aid in propagating the ultrasound signals without any refraction or minimal refraction of the ultrasound signals. Generally, the conventional probes entail use of the acoustic lenses and the front face materials. The acoustic lenses and the front face materials may have a speed of sound that is different from a speed of sound in the subject or water. Consequently, when the ultrasound signals propagate through these acoustic lenses and the front face materials, the ultrasound signals may be refracted and hence may interfere with a predetermined focusing and steering of the ultrasound signals.

To overcome the above problems, in the exemplary probe 200, the acoustic standoff 216 having the non-focusing materials is used to avoid any refraction of the ultrasound signals. Particularly, the acoustic standoff 216 includes the non-focusing materials having a speed of sound that is similar to a speed of sound in the subject or water. This in turn aids in propagating the ultrasound signals without any refraction or minimal refraction. It may be noted that the property of the acoustic standoff 216 that facilitates propagation of the ultrasound signals without any refraction or minimal refraction is referred to as “non-focusing” of the ultrasound signals. Also, it may be noted that the terms “speed of sound” and “acoustic velocity” may be used interchangeably. In one embodiment, the one or more non-focusing materials in the acoustic standoff 216 may have an acoustic velocity of about 1540 m/sec. This value of the acoustic velocity is substantially equal to a value of the acoustic velocity in the subject or water. In one example, the acoustic standoff 216 may include a TPE (Thermo Plastic Elastomer) material, a synthetic material, or any other suitable soft materials that provide low attenuation and non-focusing of the ultrasound signals.

In another embodiment, the acoustic standoff 216 may be a water-like medium, such as an acoustic coupling gel or water contained within a deformable container that may be molded or deformed to a desired shape. This acoustic standoff 216 may be disposed between the curved surface 206 of the housing 202 and the transducer unit 210. In one example, the water-like medium may be a compliant oleophilic gel that is configured to drape and conform to contours of the subject to provide enhanced imaging of an underlying target volume. Also, this water-like medium is capable of supporting longitudinal waves with minimal acoustic attenuation.

Furthermore, the acoustic standoff 216 may act as a thermal insulator between the transducer unit 210 and the subject, which in turn prevents or limits generation of a hot spot in the transducer probe 200. Also, the acoustic standoff 216 may have an impedance that is similar to an impedance of water and/or body tissue. As a result, the ultrasound signals may experience minimal or no reflection while propagating into the target volume 101 in the subject. This in turn aids in obtaining a high quality image of the target volume 101.

Moreover, in one embodiment, the acoustic standoff 216 may have an acoustic velocity in a range from about 1.03 km/sec to about 1.63 km/sec. Also, the acoustic standoff 216 may have an impedance that is in a range from about 1.43 MRayls to about 1.66 MRayls. Further, the acoustic standoff 216 may have an attenuation that is in a range from about 0.28 db/mm to about 2.80 db/mm. In addition, the acoustic standoff 216 may include one or more materials having a density in a range from about 0.92 g/cc to about 1.39 g/cc.

Referring to FIG. 3, a perspective view of an electro-acoustic module 300, in accordance with aspects of the present disclosure, is depicted. The electro-acoustic module 300 may be representative of an electro-acoustic module configured for use in the transducer unit 210 of FIG. 2. In a presently contemplated configuration, the electro-acoustic module 300 includes a matrix acoustic array 301, a flex interconnect 302, one or more application-specific integrated circuits (ASICs) 304, an acoustic backing 306, and a heat sink 308. The matrix acoustic array 301 is configured to transmit one or more acoustic waves towards a subject. The matrix acoustic array 301 may also receive reflected acoustic waves from the subject. These reflected acoustic waves may have a frequency in a range from about 0.5 MHz to about 25 MHz. It may be noted that the terms “acoustic waves” and “ultrasonic pulses” may be used interchangeably. In one embodiment, the matrix acoustic array 301 includes a single row of electrically and acoustically isolated acoustic elements. In other embodiments, the matrix acoustic array 301 may include a plurality of rows of electrically and acoustically isolated acoustic elements. Each of these acoustic elements may be a layered structure including at least a piezoelectric layer and an acoustic matching layer. In one embodiment, the matrix acoustic array 301 may include micromachined ultrasound transducers, such as capacitive micromachined ultrasonic transducers (cMUTs) and/or piezoelectric micromachined ultrasonic transducers (pMUTs).

As will be appreciated, an electrical pulse is applied to electrodes of the piezoelectric layer of the acoustic element, thereby causing a mechanical change in one or more dimensions of the piezoelectric layer. This in turn generates an acoustic wave that is transmitted towards a target volume in the subject. Further, when the acoustic waves are reflected from the target volume in the subject, a voltage difference is generated across the electrodes. This voltage difference is detected as a received signal. Received signals corresponding to each of the acoustic elements in the matrix acoustic array 301 may be combined and processed by the ASIC 304 to generate an ultrasound image of the subject.

Moreover, the matrix acoustic array 301 is coupled to the flex interconnect 302. The flex interconnect is used for providing an electrical connection between the matrix acoustic array 301 and signal processing electronics or circuit board (not shown in FIG. 3) that may be disposed within a body of a transducer probe such as the transducer probe 200 (see FIG. 2) or external to the transducer probe. In one example, the flex interconnect 302 may be used to communicate the electrical pulses between the piezoelectric layer of the acoustic elements in the matrix acoustic array 301 and the signal processing electronics.

Further, the ASIC 304 is coupled to the acoustic backing 306 and the heat sink 308, as depicted in FIG. 3. The acoustic backing 306 may be configured to absorb the acoustic waves or energy that is transmitted in a direction away from the subject being scanned. It may be noted that the acoustic waves are generated by the piezoelectric layer of the acoustic elements. A portion of the generated acoustic waves may be reflected from structures or interfaces behind the matrix acoustic array 301. These acoustic waves may combine with the acoustic waves that are reflected from the target volume 101 in the subject, thereby adversely affecting the quality of the ultrasonic image of the subject.

In accordance with aspects of the present specification, this problem may be circumvented by positioning the acoustic backing 306 below the ASIC 304. This arrangement aids in attenuating or absorbing the acoustic waves that are propagated in a direction opposite the direction of the subject. In one example, the acoustic backing 306 may include acoustic backing materials that are combinations of a high-density acoustic scatterer, such as tungsten and/or a soft acoustic absorbing material, such as silicone, in a matrix of an epoxy or a polyurethane. In another example, the acoustic backing material may include an epoxy filled graphite foam, which has the added advantage of having a high thermal conductivity to draw heat away from the ASIC 304. Also, the heat sink 308 may be configured to absorb or dissipate the heat generated in the electro-acoustic module 300. In one embodiment, the heat sink 308 along with the acoustic backing 306 may be configured to absorb the heat generated in the electro-acoustic module 300.

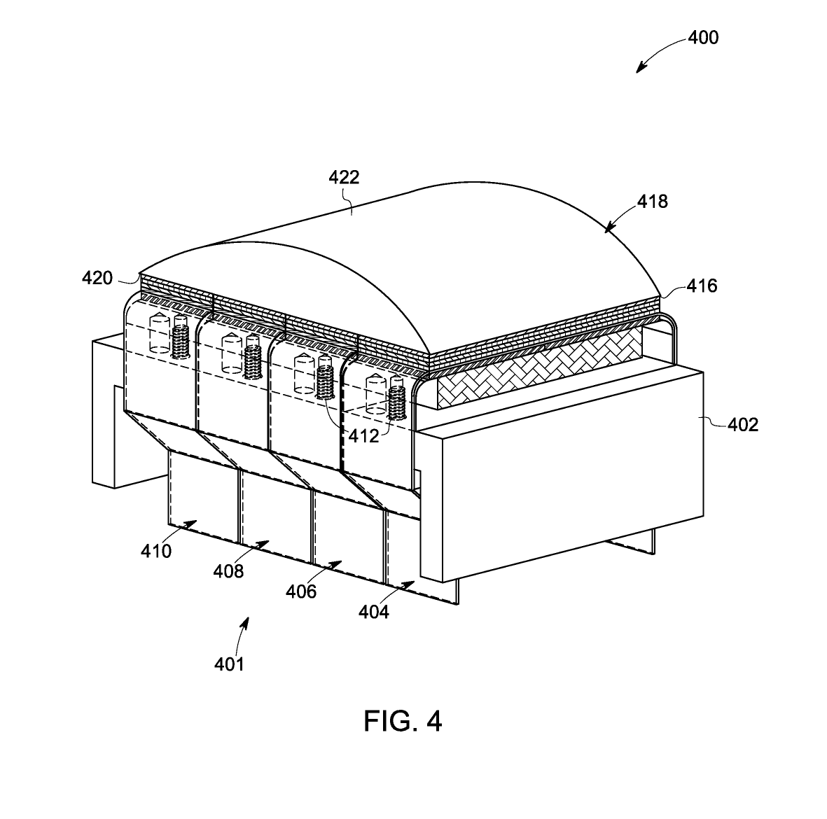

Turning now to FIG. 4, a perspective view 400 of an exemplary arrangement that includes an acoustic standoff 418 and a transducer array 401 having one or more electro-acoustic modules, is depicted. The transducer array 401 may be configured for use in the transducer unit 210 of FIG. 2. The transducer array 401 includes a support structure 402 and one or more electro-acoustic modules 404, 406, 408, 410. These electro-acoustic modules 404, 406, 408, 410 are coupled to the support structure 402. In one example, each of the electro-acoustic modules 404-410 may be coupled to the support structure 402 using fastening devices 412, as depicted in FIG. 4. It may be noted that each of these electro-acoustic modules 404-410 may be same or similar to the electro-acoustic module 300 of FIG. 3.

Further, a position of each of the electro-acoustic modules 404-410 may be interchangeable on the support structure 402. Also, each of the electro-acoustic modules 404-410 may be similar in size, which aids in easy extensibility, replaceablity, and/or flexibility during design and manufacture of an ultrasound probe such as the ultrasound probe 200 (see FIG. 2). Moreover, the electro-acoustic modules 404-410 may be tiled or aligned on the support structure 402 so as to conform to a shape of an ultrasound probe. In addition, this modular arrangement of the electro-acoustic modules 404-410 allows easy/convenient replacement of damaged electro-acoustic modules. It may be noted that the transducer array 401 may include any number of electro-acoustic modules, and is not limited to the number of electro-acoustic modules depicted in FIG. 4.

It may be noted that the arrangement of the electro-acoustic modules 404-410 on the support structure 402 results in a flat top surface 416 of the transducer array 401. Further, positioning the transducer array 401 having this flat top surface 416 below a curved surface of a housing of a probe results in a substantial air gap between the curved surface of the housing of the probe and the flat top surface 416 of the transducer array 401. This air gap may have an acoustic velocity that is substantially different from the acoustic velocity of the target volume in the subject. The difference in the acoustic velocities in turn may affect the focus of the ultrasound signals/waves and/or attenuate the ultrasound signals that are propagating from the transducer array 401 towards the target volume in the subject. Consequently, the quality of the ultrasonic image computed from these ultrasound signals may be degraded.

In accordance with aspects of the present specification, the acoustic offset or acoustic standoff 418 may be configured for use in an ultrasound probe, such as the ultrasound probe 200 of FIG. 2. Particularly, the acoustic standoff 418 may have a flat bottom surface 420 and a curved top surface 422. The flat bottom surface 420 is coupled to the flat top surface 416 of the transducer array 401, while the curved top surface 422 is coupled to the curved surface of the transducer probe. Implementing the acoustic standoff 418 in the ultrasound probe as described hereinabove advantageously results in eliminating the air gap and hence the propagation distance through air for the ultrasound signals.

Moreover, the acoustic standoff 418 may act as a non-focusing and low attenuation medium for the ultrasound signals that propagate from the transducer array 401 to the target volume. As a result, the ultrasound signals may propagate with minimal energy loss. More specifically, the acoustic standoff 418 may have an acoustic velocity that is same or substantially equal to the acoustic velocity in water, thereby minimizing any focusing of the ultrasound signals while the ultrasound signals propagate towards the target volume. Also, an impedance of the acoustic standoff 418 may match with an impedance of the target volume 101. Hence, the ultrasound signals may have minimal attenuation while propagating from the transducer array 401 to the target volume. Moreover, in situations where the ultrasound signals experience attenuation, the acoustic standoff 418 is configured to uniformly attenuate the ultrasound signals both at the middle of the acoustic standoff 418 and at the extremities of the acoustic standoff 418.

Thus, by employing the acoustic standoff 418 in the transducer probe, the ultrasound signals may be transmitted and received with negligible or no attenuation and/or focusing of the ultrasound signals. Consequently, the quality of the ultrasonic image computed using these ultrasound signals may be substantially improved.

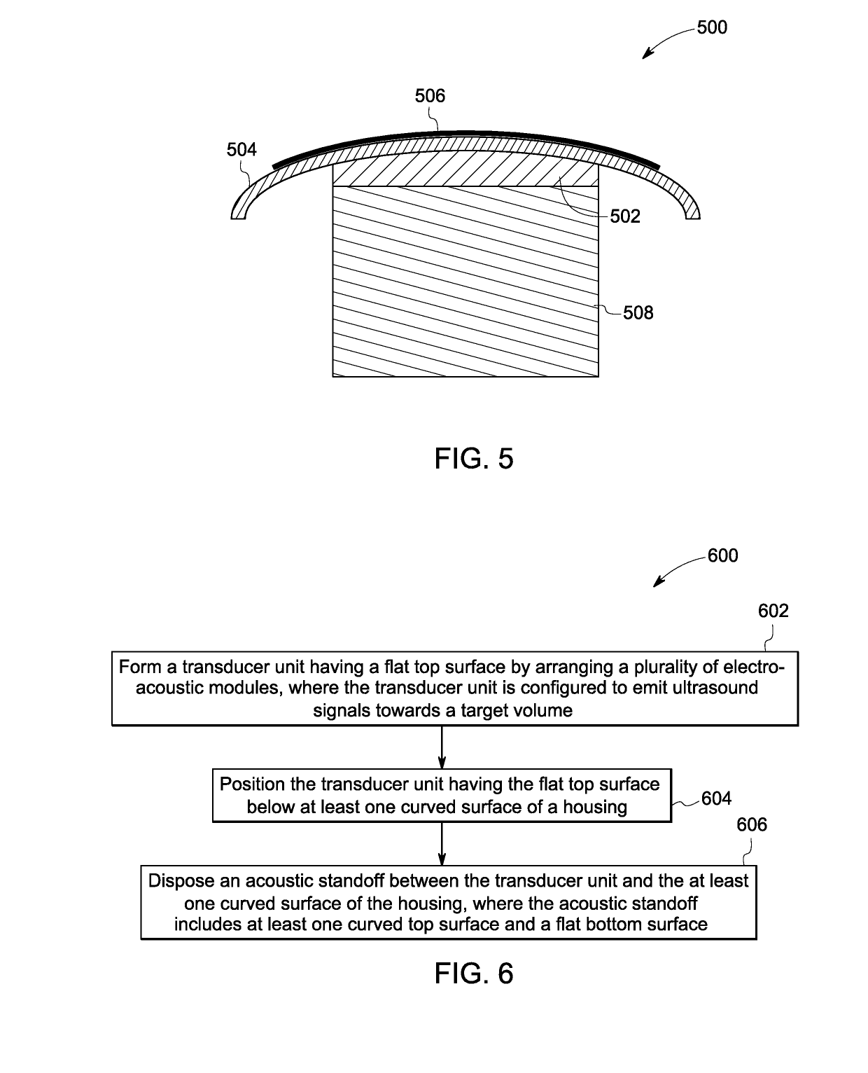

Referring to FIG. 5, a portion of a transducer probe 500 having an acoustic standoff 502, in accordance with aspects of the present specification, is depicted. The acoustic standoff 502 is disposed over a transducer unit 508. The transducer probe 500 may be similar to the transducer probe 200 of FIG. 2. However, in the embodiment of FIG. 5, the transducer probe 500 includes a cover layer 504 that is configured to protect the acoustic standoff 502 against external influences/forces. One example of the external influences/forces may include friction between the subject and the probe 500 when the probe 500 is in contact with the subject. Further, as depicted in FIG. 5, the cover layer 504 is positioned between the acoustic standoff 502 and a curved surface 506 of a housing of the transducer probe. In other embodiments, the transducer probe may not include the cover layer 504. In such embodiments, the curved surface 506 may be configured to function as the cover layer in the probe 500. Also, the cover layer 504 may have an acoustic velocity that is greater than an acoustic velocity in the acoustic standoff 502.

Moreover, in certain embodiments, the cover layer 504 may be a stiff or hard layer of material that is positioned over the acoustic standoff 502 to provide mechanical support to the acoustic standoff. In one example, the cover layer 504 may include a thermoplastic polymer material having a thickness in a range from about 0.05 mm to about 0.8 mm. However, in certain other embodiments, the curved layer 504 may be a thin protection layer that is used to mechanically and chemically protect the acoustic standoff 502. As will be appreciated, in some embodiments, the probe 500 may be a handheld device. In this example, the probe 500 is configured to be in contact with the subject during an examination of the subject. More particularly, the cover layer 504 may be positioned at a front surface of the probe 500. In such embodiments, the cover layer 504 may be configured to withstand friction and/or wear from constant contact with a surface of the subject during the examination. Also, the cover layer 504 may be configured to protect the acoustic standoff 502 and other components in the probe 500 from being damaged.

Furthermore, since the probe 500 is used in medical applications, the probe 500 may be subjected to repeated disinfection and cleaning cycles with various chemicals. Use of the cover layer 504 in the probe 500 provides protection to the acoustic standoff 502 from any direct contact with these chemicals. This in turn prevents the acoustic standoff 502 from being damaged due to exposure to the chemicals. In one embodiment, the acoustic standoff 502 acts as a bonding agent that aids in coupling or fastening the cover layer 504 to the acoustic standoff 502, thereby facilitating a tighter fit of the cover layer 504 to the acoustic standoff 502. This in turn minimizes the footprint of the transducer 500, which is particularly important for cardiac access.

FIG. 6 is a flow chart illustrating a method for forming an ultrasound probe having an exemplary acoustic standoff, in accordance with aspects of the present specification. The method is employed for forming an ultrasound transducer probe that is used for imaging a target volume, such as chest, breast, and/or abdominal regions in a patient. For ease of understanding, the method 600 is described with reference to the components of FIGS. 1-4.

The method begins at step 602, where a transducer unit 210 having a flat top surface 222 is formed. Particularly, the transducer unit 210 may be formed by tiling or aligning a plurality of electro-acoustic modules 404, 406, 408, 410 on a support structure 402. The transducer unit so formed has a flat top surface. Also, these electro-acoustic modules 404, 406, 408, 410 are used to emit ultrasound signals towards the target volume 101.

Subsequently, at step 604, the transducer unit 210 may be positioned below a curved surface 206 of a housing 202 of the transducer probe 200. As previously noted, there is an air gap between the flat top surface 222 of the transducer unit 210 and the curved surface 206 of the housing 202 of the probe 200. In accordance with exemplary aspects of the present specification, at step 606, an acoustic standoff may be disposed between the transducer unit 210 and the curved surface 206 of the housing 202. Particularly, the acoustic standoff 216 may have a flat bottom surface 218 and a curved top surface 220. The flat bottom surface 218 of the acoustic standoff 216 is coupled to the top surface 222 of the transducer unit 210, while the curved top surface 220 of the acoustic standoff 216 is coupled to the curved surface 206 of the housing 202. By placing the acoustic standoff 216 between the curved surface 206 of the housing 202 and the transducer unit 210, the air gap between the transducer unit 210 and the subject may be eliminated. Also, the acoustic standoff 216 includes one or more materials that minimize the energy loss in the ultrasound signals and/or refraction of the ultrasound signals when the ultrasound signals propagate from the transducer unit 210 to the subject.

In certain other embodiments, a cover layer 504 may be disposed between the curved surface 506 and the acoustic standoff 502, where the cover layer 504 is configured to mechanically and chemically protect the acoustic standoff 502 against external influences/forces.

The various embodiments of the exemplary system and method aid in efficiently coupling a flat transducer array to a curved probe surface. Additionally, the exemplary system and method aid in reducing the cost and complexity of fabrication of the transducer probe. Also, the exemplary system and method aid in providing ergonomic imaging capability for scanning desired volumes in the subject.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. A transducer probe, comprising:
a housing having at least one curved surface at a first end;
a transducer unit comprising a plurality of electro-acoustic modules and configured to emit ultrasound signals towards a target volume;
at least one interconnect configured to electrically couple the transducer unit to a probe cable; and
an acoustic standoff positioned between the transducer unit and the curved surface of the housing and configured to propagate the ultrasound signals with minimal attenuation, minimal refraction, or both.

2. The transducer probe of claim 1, wherein the acoustic standoff comprises a flat bottom surface and at least one curved top surface.

3. The transducer probe of claim 2, wherein the flat bottom surface of the acoustic standoff is coupled to the transducer unit and the at least one curved top surface is coupled to the at least one curved surface of the housing.

4. The transducer probe of claim 2, wherein the acoustic standoff is operatively coupled to the transducer unit via use of ultraviolet light.

5. The transducer probe of claim 2, wherein the acoustic standoff comprises one or more materials having an acoustic velocity that is equal to an acoustic velocity in water.

6. The transducer probe of claim 2, wherein the acoustic standoff comprises one or more materials having an impedance that is equal to an impedance of the target volume.

7. The transducer probe of claim 6, wherein the one or more materials of the acoustic standoff comprise at least one of a Thermo Plastic Elastomere (TPE) material and a silicon material.

8. The transducer probe of claim 2, wherein the acoustic standoff comprises an acoustic coupling gel disposed within a deformable container, and wherein the deformable container is configured to be conformable to a determined shape.

9. The transducer probe of claim 2, wherein the acoustic standoff is configured to provide thermal insulation to the transducer unit.

10. The transducer probe of claim 2, wherein a peak thickness of the acoustic standoff is in a range from about 0.5 mm to about 12 mm.

11. The transducer probe of claim 2, wherein a radius of curvature of the acoustic standoff is in a range from 9 mm to about 60 mm.

12. The transducer probe of claim 2, further comprising a cover layer disposed on the at least one curved top surface of the acoustic standoff.

13. The transducer probe of 12, wherein the acoustic standoff is a bonding agent configured to operatively couple the cover layer to the acoustic standoff.

14. The transducer probe of 12, wherein the cover layer comprises a hard layer of material configured to provide mechanical support to the acoustic standoff.

15. The transducer probe of 12, wherein the cover layer comprises a thin protection layer configured to chemically protect the acoustic standoff against one or more external influences.

16. The transducer probe of 12, wherein a thickness of the cover layer is in a range from about 0.05 mm to about 0.8 mm.

17. A system for ultrasound imaging, the system comprising:
a transmit circuitry configured to generate ultrasonic pulses;
a transducer probe coupled to the transmit circuitry via a probe cable and comprising:
a housing having at least one curved surface at a first end;
a transducer unit comprising a plurality of electro-acoustic modules configured to receive the ultrasonic pulses and emit ultrasound signals towards a target volume;
at least one interconnect configured to electrically couple the transducer unit to the probe cable; and
an acoustic standoff positioned between the transducer unit and the at least one curved surface of the housing and configured to propagate the ultrasound signals with minimal attenuation, minimal refraction, or both.

18. The system of claim 17, wherein the acoustic standoff comprises a flat bottom surface and at least one curved top surface, and wherein the flat bottom surface of the acoustic standoff is coupled to the transducer unit and the at least one curved top surface is coupled to the at least one curved surface of the housing.

19. A method for forming an ultrasound probe, the method comprising:
forming a transducer unit having a flat top surface by arranging a plurality of electro-acoustic modules, wherein the transducer unit is configured to transmit ultrasound signals towards a target volume;
positioning the transducer unit having the flat top surface below at least one curved surface of a housing; and
disposing an acoustic standoff between the transducer unit and the at least one curved surface of the housing to eliminate a distance of propagation for the ultrasound signals through air, wherein the acoustic standoff comprises at least one curved top surface and a flat bottom surface.

20. The method of claim 19, wherein disposing the acoustic standoff comprises:
coupling the flat bottom surface of the acoustic standoff to the flat top surface of the transducer unit; and
coupling the at least one curved top surface of the acoustic standoff to the at least one curved surface of the housing.
